(19) United States
(12) Patent Application Publication
(10) Pub. No.: US 2012/0123341 A1
(43) Pub. Date: May 17, 2012

(54) MONITORING SYSTEM FOR MICRONEEDLE DRUG DELIVERY

(76) Inventors: James Caradoc Birchall, Cardiff (GB); Sion Andrew Coulman, Cardiff (GB); Christopher John Allender, Cardiff (GB); Keith Roger Brain, Cardiff (GB)

(21) Appl. No.: 12/809,667

(22) PCT Filed: Dec. 18, 2008
(86) PCT No.: PCT/GB08/04201

§ 371 Date: Aug. 10, 2010

(30) Foreign Application Priority Data
Dec. 21, 2007 (GB) 0725018.6

(51) Int. Cl. A61M 5/00; B05D 3/00

(52) U.S. Cl. 604/173; 427/2.12

(57) ABSTRACT

The invention relates to a microneedle drug monitoring system, and also a microneedle device when provided with the drug monitoring system, comprising the selective and sequential coating of microneedles with layers of indicator material and therapeutic agent.

The invention relates to a system for monitoring the delivery of therapeutics by microneedles; and particularly, but not exclusively, a microneedle adapted for use in the said system.

In recent years microneedles have emerged as a technology that exists at the interface of engineering and biological sciences. They have been widely reported as an exciting alternative to conventional ‘needle and syringe’ injection. Microneedles, so termed as they generally range from 100 to 1000 μm (typically between 200 and 600 μm) in length, are designed to perforate the external skin barrier layer, the stratum corneum.

The stratum corneum provides a protective and defensive barrier that represents the upper, outermost part of the epidermis, being 10-20 μm thick. It consists of flattened corneocytes surrounded by a lipid matrix. The waterproofing barrier property of the stratum corneum is imparted by the inter-cellular multi-lamellar lipid sheets that surround individual corneocytes. The corneocytes contain densely packed insoluble keratin filaments and, although functional enzymes are present, they are regarded as non-viable due to the absence of functional organelles and their inability to regenerate. The stratum corneum is, however, in a dynamic state, with continuous renewal and modification of the extra-cellular barrier lipids and controlled desquamation of corneocytes being facilitated by a host of different enzymes.

In order to provide a direct and controlled route of access for therapeutic materials to the underlying viable tissue layers this stratum corneum must be penetrated.

In some cases microneedles can be manufactured so that the length of each microneedle is such that the depth of penetration causes minimal damage to the nerve fibres and blood vessels that reside primarily in the sub-epidermal layer, therefore, the delivery of both small and large molecular weight medicaments into the skin can be achieved without causing pain or bleeding at the site of the application.

Microneedles provide the drug-delivery specialist with a fresh opportunity for administering a range of therapeutics to, and through, skin, with the methodology conferring a number of advantages compared with alternative topical or transdermal approaches, or other physical cutaneous delivery methods. These include direct and controlled delivery of the medicament to targeted skin layers, rapid exposure of large surface areas of epidermis to the delivery agents (microneedle arrays can contain over 1000 microneedles), effortless, convenient and painless delivery for the patient, the ability to manipulate the drug formulation (e.g., solution, suspension, emulsion, dry powder and gel) for optimum effect, the use of concomitant delivery methods such as transdermal patches, and minimal invasiveness suited to patient self-administration without the need for medical supervision. A further important advantage in microneedle use lies in the ability to adapt the composition and dimensions of the needle to facilitate the delivery of a range of therapeutics including conventional drug molecules, macromolecules, nanoparticles and vaccines.

If microneedles are to be used for the transdermal delivery of medicaments currently delivered by less convenient or more invasive methods, and the microneedle structures are incorporated into a transdermal patch, it is likely that a patient would view the delivery system from a preconceived positive bias. However, it is equally important for clinicians to feel confident that the medicament to be delivered has been delivered successfully and in the correct dose. This is particularly the case where medicaments are used to treat serious or fatal conditions for example the medicament may comprise an important vaccine, a gene-based therapy for the treatment of, for e.g., inheritable conditions, or the delivery of cytotoxic agents for the treatment of skin cancer.

It is therefore important that a microneedle drug delivery system includes a monitoring means for establishing that the drug of interest has been delivered effectively into skin, i.e. to the target site, at the approximate dosage. In the case of microneedles comprising a medicament on the external, or internal, surface this effectively means that the microneedles have been left in position for the appropriate time period to allow de-coating, or release, of the medicament and dispersion into the surrounding skin compartments. Moreover, it is important that such a means is of a simple, reliable and inexpensive nature so that it can be universally used in all microneedle technologies.

With this in mind, we have designed a monitoring system that enables therapeutic delivery via microneedles to be monitored:

According to a first aspect of the invention there is provided a microneedle drug monitoring system for monitoring the effective delivery of a therapeutic agent to its target site comprising a plurality of microneedles provided on, or integral with, a microneedle substrate support characterised in that a majority of said microneedles have at least one coat of indicator material and a neighbouring at least one coat of therapeutic agent wherein said coats are arranged so that delivery of the therapeutic agent occurs prior to delivery of the indicator material whereby the indicator serves as a way of establishing that the therapeutic agent has been successfully delivered.

In a preferred embodiment of the invention, ideally, all of the said microneedles are provided with said two different coats but this is not compulsory since if enough of the needles are coated in this fashion then one can establish that enough of the therapeutic agent has been delivered to its target site.

In an alternative embodiment of the invention an indicator material is added to the drug itself to provide one coating of drug/indicator, or, in certain embodiments, multiple coatings of drug/indicator.

Reference herein to a target site includes reference to the site that the therapeutic agent is to be delivered to which may or may not include its actual site of therapeutic activity.

In a preferred embodiment of the invention said indicator material and said therapeutic agent have migratory or diffusable properties and so can move from said microneedle to said target site.

In a further preferred embodiment of the invention, where the indicator is not included in the drug, said indicator material is provided underneath the therapeutic agent as a first underlying layer. However, in an alternative embodiment said indicator material may be provided away from the tip of the microneedle and said therapeutic agent may be provided towards the tip of the microneedle so that the indicator is provided as a layer behind, or distal from, the tip of the microneedle so establishing that the therapeutic agent, located at or near the tip, has been delivered to its target site in advance of the indicator.

In yet a further preferred embodiment of the invention additional layers of indicator material and therapeutic agent may be used and therefore, in one embodiment, the microneedles may be coated with a plurality of layers of indicator materials and therapeutic agents. In this embodiment the indicator materials will be, ideally, distinguishable from each other in order to enable an observer to monitor the delivery of each of the said therapeutic agents. In this embodiment of the invention the therapeutic agents may either be distinguishable from each other or may be identical. For example in the former instance it may be advantageous to monitor the delivery of a number of different therapeutic agents. Alternatively, in the latter instance, there may be a requirement to deliver two doses of a given therapeutic agent in a delayed or timed fashion and thus the indicators serve to establish when a first dose has been delivered and, thereafter, when a further dose has been delivered.

In this layered embodiment of the invention the indicator materials and the therapeutic agents may be layered, in alternating fashion, one on top of the other. Alternatively, the indicator materials and therapeutic agents may be deposited one after another from one end of the needle to the other end of the needle.

It is possible, where an indicator material has a relatively shortly life span to use the same indicator in relation to the delivery of more than one therapeutic agent or to deliver a single therapeutic agent multiple times. In this instance, the observer would be looking for the appearance of the indicator at the target site, its subsequent disappearance and then its appearance at the target site following the delivery of the further therapeutic.

Preferred indicator materials for use in the invention comprise visible indicators such as inert physiological dyes all of which are well known to those skilled in the art but a preferred dye is methylene blue. Further indicator materials for use in the invention are described on pages 10 and 11.

According to a further aspect of the invention there is provided a plurality of microneedles attached to a common substrate wherein the majority of said microneedles have coated thereon either alternating layers of indicator material and therapeutic agent or multiple layers of drugs where each drug layer includes or has incorporated therein a selected indicator.

In this preferred embodiment of the invention the layers may be adjacent to one another or on top of one another.

In the instance where each drug layer includes, or has incorporated therein, a selected indicator hollow microneedles may be used and each drug is layered one behind the other inside the needle.

According to a yet further aspect of the invention there is provided a method for the manufacture of a microneedle array for use in monitoring the delivery of therapeutic agents wherein the method comprises:

(a) the selective coating of at least a first part of a microneedle, and preferably all the microneedles in the array, with a first indicator material;
(b) the selective coating of either said first part or a different second part of said indicator coated microneedle with a therapeutic agent; and
(c) optionally, the repetition of parts (a) and/or (b) until said microneedle(s) have been coated with the requisite number of indicator material layers and therapeutic agent layers to enable the effective monitoring of selected therapeutic agent layer to its target site.

Further the invention extends to a microneedle drug monitoring system for monitoring the effective delivery of a therapeutic agent to its target site comprising a plurality of microneedles provided on, or integral with, a microneedle substrate support characterised in that a first group of said microneedles have at least one coat of a first indicator material and a neighbouring at least one coat of a first therapeutic agent wherein said coats are arranged so that delivery of the therapeutic agent occurs prior to delivery of the indicator material whereby the indicator serves as a way of establishing that the therapeutic agent has been successfully delivered.

In an embodiment said plurality of needles includes at least a second group having a coat of a second indicator material and a neighbouring coat of a second therapeutic agent, said second indicator material and/or said second agent being different to the first respective material or agent.

An embodiment of the invention will now be described by way of example only with reference to the following Figures wherein:

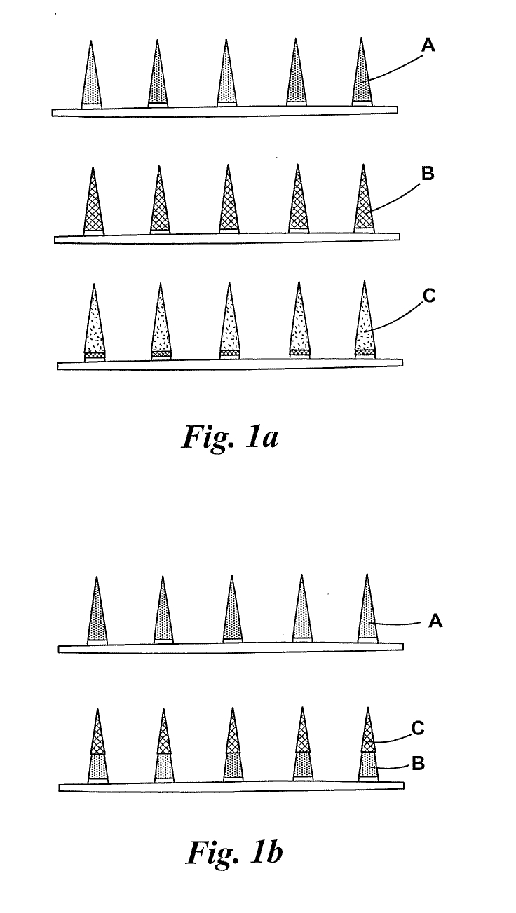

FIGS. 1a and 1b show a diagrammatic representations of microneedles coated with a single layer of indicator material and a single layer of therapeutic agent;

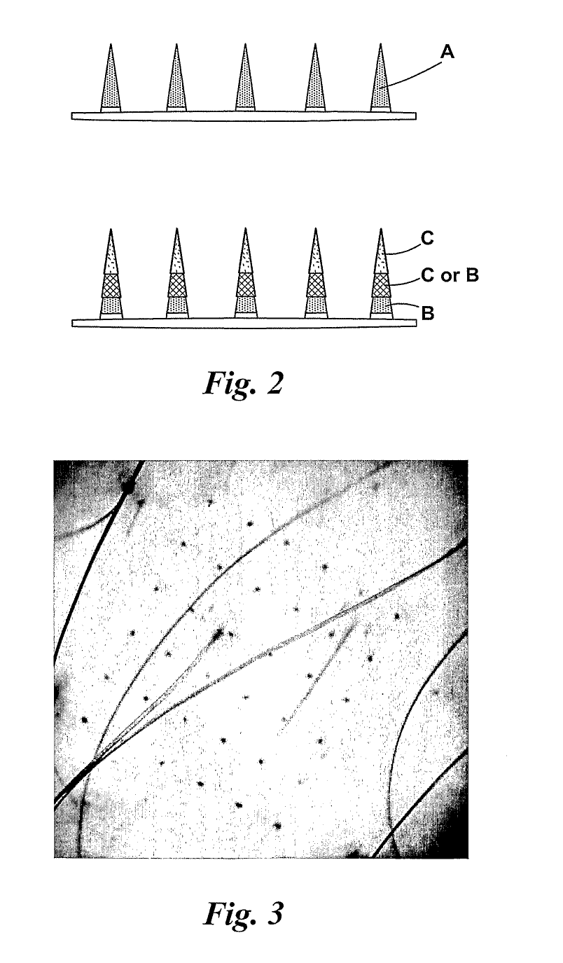

FIG. 2 shows a diagrammatic illustration of microneedles coated, in an alternative fashion, with a single layer of indicator and a single layer of therapeutic;

FIG. 3 shows methylene blue staining of microchannels in human subjects.

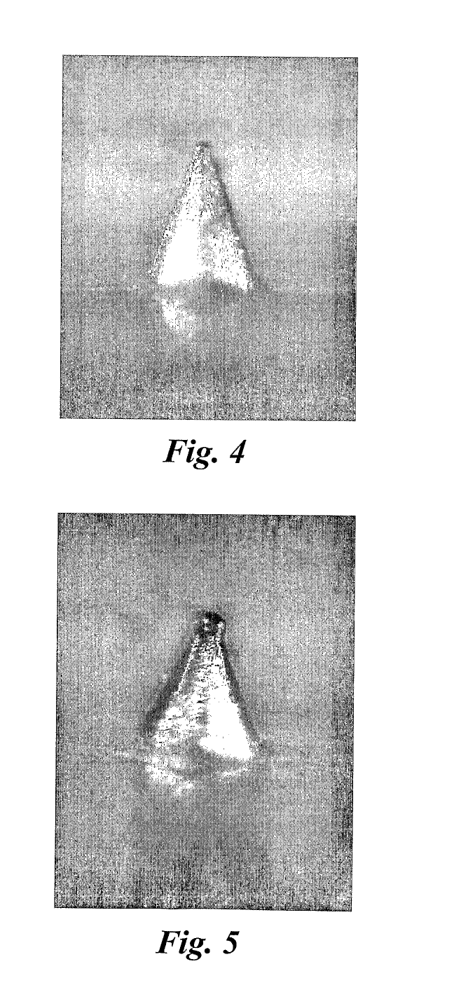

FIG. 4 shows microneedles coated with an indicator material (methylene blue);

FIG. 5 shows microneedles coated with another indicator material (nuclear fast red).

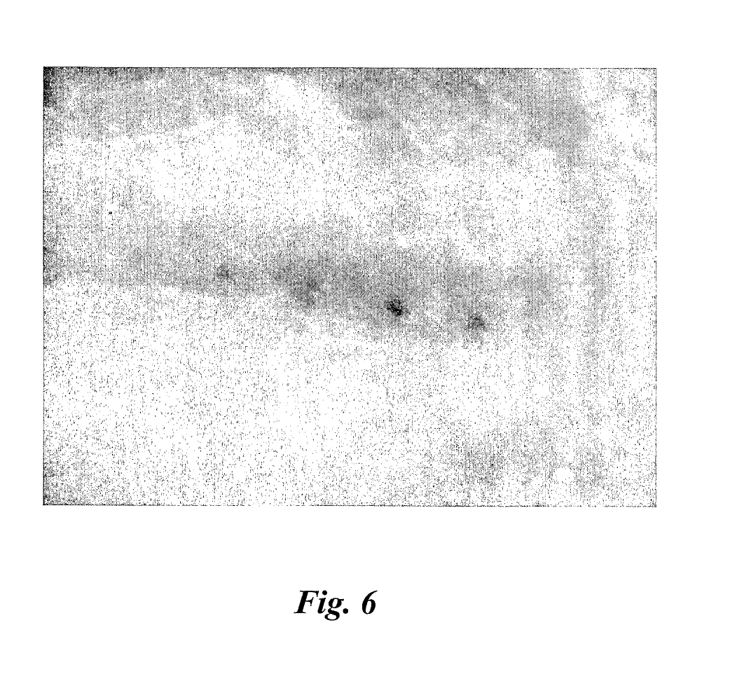

FIG. 6 shows methylene blue staining of skin following the insertion and removal of microneedles;

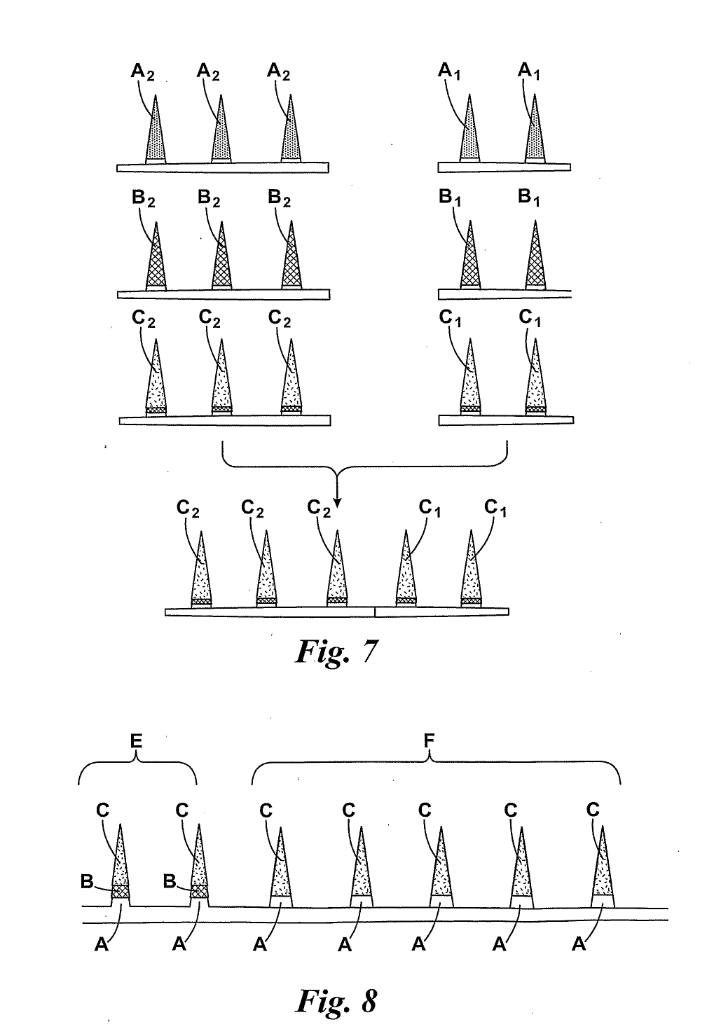

FIG. 7 shows two groups of microneedles; and

FIG. 8 shows an alternative array of microneedles coated in a different.

A microneedle array was either prepared in a conventional fashion or obtained from an available source.

In the former instance the microneedle array, involved silicon microfabrication using an etching process, either wet (solution) or dry (gas) to specifically remove predefined areas of silicon surface from a flat platform to leave needle-shaped islands. Alternatively, dry-etched fabrication of microneedle devices from silicon wafers uses a lithographically patterned mass and a blend of reactive ion gases. However, microneedle devices can be obtained from suppliers including Silex Microsystems AB, Sweden and Dermaroller S.A.R.L., France.

Typically, microneedles are coated into a fluid to be deposited thereon and then, depending upon the nature of the fluid, left to dry.

In the present invention, a microneedle array is firstly coated with a first indicator solution of physiologically inactive dye such as methylene blue or nuclear fast red or Evans Blue or Gention Violet. Alternatively food colourings can be used such as any one or more of the following approved food colourings FD&C Blue No. 1—Brilliant Blue FCF, E133 (Blue shade), FD&C Blue No. 2—Indigotine, E132 (Dark Blue shade), FD&C Green No. 3—Fast Green FCF, E143 (Bluish green shade), FD&C Red No. 40—Allura Red AC, E129 (Red shade), FD&C Red No. 3—Erythrosine, E127 (Pink shade) [4], FD&C Yellow No. 5—Tartrazine, E102 (Yellow shade), FD&C Yellow No. 6—Sunset Yellow FCF, E110 (Orange shade), alternatively, other dyes may be considered for use such as those that are used in face painting or tattooing. Those skilled in the art will appreciate that the choice of indicator will be chosen having regard to its availability, ease of use and cost.

Once the array has been coated in a first indicator, the array is left to dry for 24 hours and then coated into a solution of therapeutic agent at the required concentration before again, being left to dry for a further 24 hours.

The formulation used to promote coating of both the indicator and the therapeutic can be modified for optimised viscosity and surface activity. Example viscosity enhancers include acacia, tragacanth, alginic acid, carrageenan, locust bean gum, guar gum, gelatine, methylcellulose, sodium carboxymethylcellulose, Carbopol®, bentonite and Veegum®. Example surface active agents include sodium dodecyl sulfate (SDS), ammonium lauryl sulfate, and other alkyl sulfate salts, sodium laureth sulfate, alkyl benzene sulfonate, soaps or fatty acid salts, cetyl trimethylammonium bromide (CTAB) and other alkyltrimethylammonium salts, cetylpyridinium chloride (CPC) polyethoxylated tallow amine (POEA), benzalkonium chloride (BAC), benzethonium chloride (BZT), dodecyl betaine, dodecyl dimethylamine oxide, cocamidopropyl betaine, coco ampho glycinate, alkyl poly(ethylene oxide), copolymers of poly(ethylene oxide) and poly(propylene oxide) (commercially called Poloxamers or Poloxamines) and alkyl polyglucosides.

These additional agents are used at a concentration of 0.1-50% w/v and, ideally, 0.5-5% w/v.

This layering of one coat upon another is shown in FIG. 1a where microneedles (A) are coated with an indicator in the form of a coloured dye (B) and a further coating of therapeutic agent (C).

In FIG. 1b microneedles (A) are coated with a dye (B) then over-coated partially with a therapeutic agent (C).

In an alternative embodiment of the invention, as shown in FIG. 2, the coating of the microneedles occurs in a lateral fashion, rather than an overlapping fashion, and therefore a first end of the microneedles (A) is coated with coloured dye (B) and another end of the microneedles (A) is coated with a therapeutic agent (C).

Once the coated microneedles have been prepared they can then be used in conventional fashion and thus they are typically applied, for example on a transdermal patch, with the microneedles pressing into the skin, in a conventional fashion by the application of a suitable force. This force pushes the microneedles into the stratum corneum and allows for the diffusion of, firstly, the therapeutic and then the indicator material into skin tissue.

FIG. 3 shows methylene blue staining of microchannels produced as a result of the visible indicator travelling through skin tissue.

FIGS. 4 and 5 show images of microneedles where the microneedles have been coated with a first therapeutic agent (not shown) and overlying same is a layer of indicator material (shown). In FIG. 4 the indicator material is methylene blue and in FIG. 5 the indicator material is nuclear fast red.

FIG. 6 shows a line of blue staining following insertion and removal of double coated microneedles. The image clearly shows the presence of blue colouration indicating that, firstly, the overlying ‘drug’ layer and, secondly, the underlying visible indicator layer have de-coated from the microneedles and delivered the therapeutic into human skin.

Although the invention has been exemplified with reference to a visible indicator, as previously mentioned, a non-visible indicator can be used such as an ultra-violet reflective stain which is thus only visible upon exposure to ultra-violet, fluorescent light or other electromagnetic radiation. This sort of indicator may be preferred by users because it does not leave an unsightly stain on the skin. Nanoparticles which fluoresce under the influence of electromagnetic radiation could be employed with equal effect.

Although not visible in the drawings, as described above, microneedles may be coated with more than one layer of indicator material and also with more than one layer of therapeutic agent and, in this instance, layers of indicator material and therapeutic agent will be sequentially applied to selected parts of the microneedles in order to build overlying or adjacent layers of indicator/therapeutic.

The coating of the needles can be carried out by means of dip coating. In a further alternative the coating process, both for indicator materials and the therapeutic agent, can be achieved by aerosolising those materials and spraying them onto the needles, with or without appropriate masking. In this respect the disclosure in GB 0725017.8 is incorporated herein by reference.

In a further alternative, different needles can be coated with different indicator materials. So for example where a first therapeutic agent is applied to one group of needles and a second agent is applied to a second group in the same array, then possibly different colour indicators can be used for each group, for example were the first and second agents have different absorption properties or viscosities. FIG. 7 illustrates two such groups denoted by the suffix 1 and 2, where construction and coating of the microneedle arrays is the same as detailed above except that group 1 is formed separately from group 2 and their respective substrates are brought together at a joint (D) after appropriate coating. Other group arrangements are possible and multiple groups can be employed.

In yet another alternative a majority of microneedles can be coated with a therapeutic agent, and a minority can then be coated, with indicator material, either as an coating over/next to the agent or on separate needles. This alternative can be employed where the agent is particularly sensitive to, or interacts with the indictor, and so the agent's effect will not be significantly altered because the agent will either not come into contact with the indicator material, or may come into contact only at limited sites where the indicator material has been used. This limited use of indicator material will be of benefit also where temporary skin staining is to be minimised, for example for cosmetic reasons. Such an alternative is shown in FIG. 8 where microneedles (A) have been selectively coated in one location (E) with an indicator (B) and then a therapeutic agent (C), and at another location (F) only with a therapeutic agent (C). In this example the number of needles having only the therapeutic agent (C) coating is more than number of needles having the additional indicator material coating (B).

The technology provides for the effective monitoring of the safe delivery of a therapeutic agent(s) to its target sight.

1. A microneedle drug monitoring system for monitoring the effective delivery of a therapeutic agent to its target site comprising a plurality of microneedles provided on, or integral with, a microneedle substrate support characterised in that a majority of said microneedles have at least one coat of indicator material and a neighbouring at least one coat of therapeutic agent wherein the said coats are arranged so that delivery of the therapeutic agent occurs prior to delivery of the indicator material whereby the indicator serves as a way of establishing that the therapeutic agent has been successfully delivered.

2. A monitoring system according to claim 1 wherein all of said microneedles are provided with said two different coats.

3. A monitoring system according to claim 1 wherein said indicator material and said therapeutic agent have migratory or diffusable properties and so can move from said microneedle to said target site.

4. A monitoring system according to any preceding, claim wherein said indicator material is provided underneath the therapeutic agent as an underlying layer.

5. A monitoring system according to claim 1 wherein said indicator material is provided away from the tip of the microneedle and said therapeutic agent is provided towards the tip of the microneedle.

6. A monitoring system according to claim 1 wherein additional layers of indicator material and therapeutic agent are used whereby the microneedles are coated with a plurality of layers of indicator materials and therapeutic agents.

7. A monitoring system according to claim 6 wherein the indicator materials are distinguishable from each other.

8. A monitoring system according to claim 6 wherein the therapeutic agents may either be distinguishable from each other or identical.

9. A monitoring system according to claim 1 wherein the indicator materials and the therapeutic agents are layered, in alternating fashion, one on top of the other.

10. A monitoring system according to claim 1 wherein the indicator materials and therapeutic agents are deposited one after another from one end of the needle, i.e. the tip, to the other end of the needle.

11. A monitoring system according to claim 1 wherein the indicator material is a visible indicator.

12. A microneedle delivery device comprising a plurality of microneedles attached to a common substrate wherein the majority of said microneedles have coated thereon either alternating layers of indicator material and therapeutic agent or multiple layers of drugs wherein each of said drug layer includes, or has incorporated therein, a selected indicator.

13. A device according to claim 12 wherein the layers may be adjacent to one another or on top of one another.

14. A method for the manufacture of a microneedle array for use in monitoring the delivery of therapeutic agents wherein the method comprises:
(a) the selective coating of at least a first part of a microneedle, and preferably all the microneedles in the array, with a first indicator material;
(b) the selective coating of either said first part or a different second part of said indicator coated microneedle with a therapeutic agent: and (c) optionally, the repetition of parts (a) and/or (b) until the said microneedle(s) have been coated with the requisite number of indicator material layers and therapeutic agent layers to enable the effective monitoring of selected therapeutic agent layer to its target site.

15. A microneedle drug monitoring system for monitoring the effective delivery of a therapeutic agent to its target site comprising a plurality of microneedles provided on, or integral with, a microneedle substrate support characterised in that a first group of said microneedles have at least one coat of a first indicator material and a neighbouring at least one coat of a first therapeutic agent wherein said coats are arranged so that delivery of the therapeutic agent occurs prior to delivery of the indicator material whereby the indicator serves as a way of establishing that the therapeutic agent has been successfully delivered.

16. A monitoring system according to claim 15 wherein said plurality of needles includes at least a second group having a coat of a second indicator material and a neighbouring coat of a second therapeutic agent, second indicator material and/or said second agent being different to the first respective material or agent.

17. A monitoring system according to claim 1 wherein the indicator material is selected from any one or more of following: methylene blue, nuclear fast red, Evans Blue, Gention Violet, a food colouring such as FD&C Blue No. 1—Brilliant Blue FCF, E133 (Blue shade), FD&C Blue No. 2—Indigotine, E132 (Dark Blue shade), FD&C Green No. 3—Fast Green FCF, E143 (Bluish green shade), FD&C Red No. 40—Allura Red AC, E129 (Red shade), FD&C Red No. 3—Erythrosine, E127 (Pink shade) [4], FD&C Yellow No. 5—Tartrazine, E102 (Yellow shade), FD&C Yellow No. 6—Sunset Yellow FCF, E110 (Orange shade), face painting dyes, tattooing dyes, fluorescent markers.

18. A monitoring system as claimed in claim 17, wherein said indicator material includes a viscosity enhancers and/or a surface active agent.

19. A microneedle delivery device according to claim 12 wherein the indicator material is selected from any one or more of following: methylene blue, nuclear fast red, Evans Blue, Gention Violet, a food colouring such as FD&C Blue No. 1—Brilliant Blue FCF, E133 (Blue shade), FD&C Blue No. 2—Indigotine, E132 (Dark Blue shade), FD&C Green No. 3—Fast Green FCF, E143 (Bluish green shade), FD&C Red No. 40—Allura Red AC, E129 (Red shade), FD&C Red No. 3—Erythrosine, E127 (Pink shade) [4], FD&C Yellow No. 5—Tartrazine, E102 (Yellow shade), FD&C Yellow No. 6—Sunset Yellow FCF, E110 (Orange shade), face painting dyes, tattooing dyes, fluorescent markers.

20. A microneedle delivery device as claimed in claim 19, wherein said indicator material includes a viscosity enhancers and/or a surface active agent.
